(19) United States
(12) Patent Application Publication
(10) Pub. No.: US 2024/0398707 A1
(43) Pub. Date: Dec. 5, 2024

(54) Biocompatible And Allergen Free Lipid Emulsions

(71) Applicant: Bard Peripheral Vascular, Inc., Franklin Lakes, NJ (US)

(72) Inventor: Hiep Do, Chandler, AZ (US)

(73) Assignee: Bard Peripheral Vascular, Inc., Franklin Lakes, NJ (US)

(21) Appl. No.: 18/691,309

(22) PCT Filed: Oct. 7, 2022
(86) PCT No.: PCT/US2022/077727

§ 371 Date: Mar. 12, 2024

Related U.S. Application Data
(60) Provisional application No. 63/253,929, filed on Oct. 8, 2021.

(51) Int. Cl. A61K 9/107; A61K 47/24; A61K 47/26; A61K 47/44

(52) U.S. Cl. CPC A61K 9/1075; A61K 47/24; A61K 47/26; A61K 47/44

(57) ABSTRACT

A lipid emulsion is provided herein, including sunflower lecithin, an oil, a co-surfactant, and a cryogenic agent. Also provided is a method of preparing a lipid emulsion, the method including: (i) dissolving sunflower lecithin in water to create an aqueous phase; (ii) mixing an oil, a co-surfactant and a cryogenic agent to create an organic phase; and (iii) homogenizing the aqueous and organic phases to produce the lipid emulsion, wherein the lipid emulsion has a mean particle size of less than about 1000 nm.

### TECHNICAL FIELD

The present specification generally relates to biocompatible lipid emulsions, and, more specifically, lipid emulsions for parenteral administration, including during endoscopic treatment, as well as methods for preparing the biocompatible lipid emulsions.

### CROSS-REFERENCE TO RELATED APPLICATIONS

This application claims priority to U.S. Provisional Patent Application Ser. No. 63/253,929, filed Oct. 8, 2021, which is incorporated by reference herein in its entirety.

### BACKGROUND

Lipid emulsions have a variety of pharmacological uses. Lipid pharmaceutical formulations often are administered topically or gastrointestinally. However, lipid emulsions are administered also via a variety of parenteral routes (e.g., injectable, intravenous, ocular, epidural, etc.). For example, lipid emulsions may be infused into patients intravenously for nutrition purposes. Examples of such intravenously infused lipid emulsions include Intralipid® and Liposyn®. Lipid emulsions may be used also as a drug delivery medium. For example, Diprivan®, containing propofol, and Diazemuls®, containing diazepam, comprise lipid emulsions that aid in delivery of the active agents.

Lipid emulsions may be used also as lubricants for various medical procedures (e.g., endoscopic procedures, intravascular procedures, etc.). During procedures, lubricant may be added to allow a clinician to pass the medical device into a body lumen more easily. Lubrication may be added during these procedures also to cool medical devices from the heat generated by mechanical shear force during operation. In addition to performing a cooling function, additional lubrication may be needed to reduce friction and wear on the device. Factors that can influence friction or wear include temperature, speed, material of the device, contact area, time, and the type and amount of the lubricant. During operation of the device, additional lubrication should be provided to sustain the device's performance for the duration of the procedure. During these procedures, lubricant is infused into the patient, such that a biocompatible lubricant may be desired.

Emulsions tend to be thermodynamically unstable formulations and will undergo physical changes over time, including for example aggregation or formation of large droplets of non-emulsified oil on the surface (e.g., creaming, and droplet size increase). These changes may begin to occur within the first hour following dilution and may be accelerated by heating or by applying any shear force. Emulsifiers are added to stabilize emulsions by reducing interfacial tension of the system and by providing enough surface charge for droplet-droplet repulsion. Natural lecithin, obtained from egg yolk, has been used extensively to stabilize injectable emulsions. These emulsifiers are biocompatible, nontoxic, and metabolized easily by the body. However, allergy to eggs is one of the three most common food allergies. Emulsions containing egg yolk lecithin are therefore contraindicated for use in a substantial portion of the population. Additionally, hydrolysis of egg yolk lecithin during emulsification, sterilization and storage can lead to the formation of lysophospholipids, which can lead to membrane structural damage and hemolysis.

Accordingly, a need exists for biocompatible lipid emulsions that are allergen-free and demonstrate increased stability as well as methods of making said lipid emulsions.

### SUMMARY

It is understood that both the following summary and the detailed description are exemplary and explanatory and are intended to provide further explanation of the disclosure as claimed. Neither the summary nor the description that follows is intended to define or limit the scope of the disclosure to the particular features mentioned in the summary or description.

Embodiments as described herein generally relate to biocompatible and allergen-free lipid emulsions suitable for parenteral administration into a subject or patient. Generally, an emulsion is a mixture of two immiscible liquids, wherein one of the liquids is dispersed in the other as small droplets or particles. In embodiments, the lipid emulsion is an oil-in-water emulsion and is prepared by homogenizing organic and aqueous phases. In embodiments, the lubricant includes an oil, a surfactant, a co-surfactant, and water. The lubricant may also include a cryogenic agent, a preservative and/or a buffer. In embodiments, the lipid emulsion may have a mean particle or droplet diameter of less than about 1000 nanometers. In particular, the lipid emulsion may have a mean particle or droplet diameter of less than about 500 nanometers. The emulsion shows increased stability by enduring substantial mechanical shear forces, exhibiting suitable ambient shelf life, and tolerating freeze-thaw cycles.

In a first aspect, the present disclosure relates to parenteral lipid emulsions comprising from about 0.3 g/100 mL to about 3 g/100 mL sunflower lecithin, from about 1 g/100 mL to about 40 g/100 mL oil, and from about 0.1 g/100 mL to about 10 g/100 mL co-surfactant. The lipid emulsion also includes from about 0.1 g/100 mL to about 10 g/100 mL cryogenic agent and an aqueous liquid. The oil is selected from almond oil, canola oil, castor oil, corn oil, cottonseed oil, olive oil, peanut oil, safflower oil, soybean oil, sunflower oil, sesame oil, or mixtures thereof. The co-surfactant is selected from sodium deoxycholate, sorbitan monolaurate, sorbitan monostearate, sorbitan tristearate, polysorbate 20, polysorbate 40, polysorbate 60, polysorbate 80, poloxamers, or mixtures thereof. The cryogenic agent is selected from glycerin, DMSO, ethylene glycol, propylene glycol, sucrose, trehalose, or mixtures thereof. The emulsion may further include from about 0.001 g/100 mL to about 0.1 g/100 mL of an antimicrobial preservative selected from disodium EDTA, tocopherol, butylated hydroxyanisole (BHA), ascorbic acid, butylated hydroxytoluene (BHT), or a mixture thereof. The emulsion may have a mean particle diameter of less than 1000 nm.

A further aspect relates to the preparation of parenteral lipid emulsions as described hereinabove, comprising the steps of dissolving sunflower lecithin in water to create an aqueous phase, mixing an oil, a co-surfactant, and a cryogenic agent to create an organic phase and homogenizing the aqueous and organic phases to produce the lipid emulsion.

Yet a further aspect relates to the lipid emulsions as described hereinabove, for use in surgery, for use in therapeutic administration, or for use as a lubricant with medical devices, such as atherectomy devices.

These and additional features provided by the embodiments described herein will be more fully understood in view of the following detailed description.

### DETAILED DESCRIPTION

The details of one or more embodiments of the presently disclosed subject matter are set forth in this document. The following description of particular aspect(s) is merely exemplary in nature and is in no way intended to limit the scope of the present disclosure, its application, or its uses, all of which may vary. The present disclosure is described with relation to the non-limiting definitions and terminology included herein. These definitions and terminology are not designed to function as a limitation on the scope or practice of the present disclosure but are presented for illustrative and descriptive purposes only. While the compositions or processes are described as using specific materials or an order of individual steps, it is to be appreciated that materials or steps may be interchangeable such that the description of the present disclosure may include multiple parts or steps arranged in many ways readily appreciated by one of skill in the art. Modifications to embodiments described in this document, and other embodiments, will be evident to those of ordinary skill in the art after a study of the information provided in this document.

The terminology used herein is for describing particular aspect only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject matter. When referring to a value or to an amount of mass, weight, time, volume, concentration or percentage, the term “about” is meant to encompass variations from the specified amount of in some embodiments ±20%, in some embodiments ±10%, in some embodiments ±5%, in some embodiments ±1%, in some embodiments ±0.5%, and in some embodiments ±0.1%, provided that such variations remain appropriate to perform the disclosed method.

It should be understood that every maximum numerical limitation disclosed in this specification includes every lower numerical limitation, as though such lower numerical limitations were expressly disclosed herein. Every minimum numerical limitation disclosed in this specification will include every higher numerical limitation, as though such higher numerical limitations were expressly disclosed herein. Every numerical range disclosed in this specification will include every narrower numerical range that falls within such broader numerical range, as though such narrower numerical ranges were all expressly disclosed herein.

Embodiments as described herein generally relate to biocompatible and allergen-free lipid emulsions suitable for infusion into a patient or subject. In one or more embodiments, the lipid emulsion comprises a continuous aqueous phase and a non-continuous organic phase dispersed through the aqueous phase. Emulsions according to the present disclosure generally contain an aqueous solution and a lipid or oil. Suitable emulsions may have a wide range of viscosities, depending on the desired product form. In embodiments, lipid emulsion is an oil-in-water emulsion and is prepared by homogenizing organic and aqueous phases. In embodiments, the emulsion includes an oil, a surfactant, a co-surfactant, and water. The emulsion may also include a cryogenic agent, a preservative and/or a buffer.

The aqueous phase refers to the external or continuous phase of an oil-in-water emulsion. The aqueous phase of the lipid emulsion may be any suitable fluid such as water or a solution containing both water and one or more organic or inorganic compounds dissolved in the water or otherwise completely miscible with the water. In embodiments, the aqueous phase includes a water-containing liquid, which can contain pharmaceutically acceptable additives such as pH buffering, chelating, complexing and solubilizing agents, antioxidants and antimicrobial preservatives, suspending and/or viscosity modifying agents, or other biocompatible materials.

As used in the present disclosure, an emulsifier refers to a compound that decreases the surface tension between two immiscible substances. An emulsifier may comprise surfactants, detergents, wetting agents, dispersants, nanoparticles, polyacrylamides, or combinations thereof. In one or more embodiments, an emulsifying agent comprises at least one amphiphilic organic compound. Suitable emulsifiers are disclosed in, for example, McCutcheon's 2013 Emulsifiers and Detergents, North American Edition (2013).

In embodiments, emulsifiers can be cationic, anionic, or non-ionic. When charged emulsifiers coat droplets, the charges on the outside of the oil droplets electrostatically repel each other, thereby preventing coalescence. Non-ionic emulsifiers tend to have large head groups that sterically hinder coalescence. The type of emulsifier used depends on the application, with cationic emulsifiers typically used in low-to-neutral pH solutions and anionic emulsifiers in alkaline solutions. Non-ionic emulsifiers can be used alone or in combination with charged emulsifiers to increase emulsion stability.

In embodiments, emulsifiers may include biocompatible surfactants, bile salts and their derivatives. These may include, but are not limited to, deoxycholic acid, lecithin, catecholamines, beta-2 adrenergic receptor agonists (e.g., isoproterenol), alpha-2 adrenergic receptor agonists (e.g., yohimbine), phosphodiesterase inhibitors (e.g., aminophylline, theophylline), corticosteroids, caffeine, hyalorunidase, collagenase, alpha-tocopherol, ethanol, benzyl alcohol, carnitine, catechin, cysteine, gallic acid, laminarin, rutin, myrecetin, alpha melanocyte stimulating hormone (alpha MSH), melilotus, resveratrol, genistein, and the like. In embodiments, the emulsifier is a lecithin. Examples of lecithins include, but are not limited to vegetable lecithins such as sunflower lecithin, soy lecithin, and corn lecithin. In embodiments, the lecithin is present in the emulsion in a concentration from about 0.3 to about 3 g/100 mL emulsion, including about 0.4 g/100 mL, about 0.5 g/100 mL, about 0.6 g/100 mL, about 0.7 g/100 mL, about 0.8 g/100 mL, about 0.9 g/100 mL, about 1.0 g/100 mL, about 1.1 g/100 mL, about 1.2 g/100 mL, about 1.3 g/100 mL, about 1.4 g/100 mL, about 1.5 g/100 mL, about 1.6 g/100 mL, about 1.7 g/100 mL, about 1.8 g/100 mL, about 1.9 g/100 mL, about 2.0 g/100 mL, about 2.1 g/100 mL, about 2.2 g/100 mL, about 2.3 g/100 mL, about 2.4 g/100 mL, about 2.5 g/100 mL, about 2.6 g/100 mL, about 2.7 g/100 mL, about 2.8 g/100 mL, about 2.9 g/100 mL, or any range having endpoints defined by any two of the aforementioned values.

In embodiments, the lecithin is present in the emulsion in a concentration ranging from about 0.3 g/100 mL to about 2.5 g/100 mL, from about 0.4 g/100 mL to about 2.5 g/100 mL, from about 0.5 g/100 mL to about 2.5 g/100 mL, from about 0.6 g/100 mL to about 2.5 g/100 mL, from about 0.7 g/100 mL to about 2.5 g/100 mL, from about 0.8 g/100 mL to about 2.5 g/100 mL, from about 0.9 g/100 mL to about 2.5 g/100 mL, from about 1.0 g/100 mL to about 2.5 g/100 mL, from about 1.1 g/100 mL to about 2.5 g/100 mL, from about 1.2 g/100 mL to about 2.5 g/100 mL, from about 1.3 g/100 mL to about 2.5 g/100 mL, from about 1.4 g/100 mL to about 2.5 g/100 mL, from about 1.5 g/100 mL to about 2.5 g/100 mL, from about 1.6 g/100 mL to about 2.5 g/100 mL, from about 1.7 g/100 mL to about 2.5 g/100 mL, from about 1.8 g/100 mL to about 2.5 g/100 mL, from about 1.9 g/100 mL to about 2.5 g/100 mL, from about 2.0 g/100 mL to about 2.5 g/100 mL, from about 2.25 g/100 mL to about 2.5 g/100 mL, from about 0.3 g/100 mL to about 2.0 g/100 mL, from about 0.4 g/100 mL to about 2.0 g/100 mL, from about 0.5 g/100 mL to about 2.0 g/100 mL, from about 0.6 g/100 mL to about 2.0 g/100 mL, from about 0.7 g/100 mL to about 2.0 g/100 mL, from about 0.8 g/100 mL to about 2.0 g/100 mL, from about 0.9 g/100 mL to about 2.0 g/100 mL, from about 1.0 g/100 mL to about 2.0 g/100 mL, from about 1.1 g/100 mL to about 2.0 g/100 mL, from about 1.2 g/100 mL to about 2.0 g/100 mL, from about 1.3 g/100 mL to about 2.0 g/100 mL, from about 1.4 g/100 mL to about 2.0 g/100 mL, from about 1.5 g/100 mL to about 2.0 g/100 mL, from about 1.6 g/100 mL to about 2.0 g/100 mL, from about 1.7 g/100 mL to about 2.0 g/100 mL, from about 1.8 g/100 mL to about 2.0 g/100 mL, from about 1.9 g/100 mL to about 2.0 g/100 mL, from about 0.3 g/100 mL to about 1.5 g/100 mL, from about 0.4 g/100 mL to about 1.5 g/100 mL, from about 0.5 g/100 mL to about 1.5 g/100 mL, from about 0.6 g/100 mL to about 1.5 g/100 mL, from about 0.7 g/100 mL to about 1.5 g/100 mL, from about 0.8 g/100 mL to about 1.5 g/100 mL, from about 0.9 g/100 mL to about 1.5 g/100 mL, from about 1.0 g/100 mL to about 1.5 g/100 mL, from about 1.1 g/100 mL to about 1.5 g/100 mL, from about 1.2 g/100 mL to about 1.5 g/100 mL, from about 1.3 g/100 mL to about 1.5 g/100 mL, from about 1.4 g/100 mL to about 1.5 g/100 mL, from about 0.3 g/100 mL to about 1.25 g/100 mL, from about 0.4 g/100 mL to about 1.25 g/100 mL, from about 0.5 g/100 mL to about 1.25 g/100 mL, from about 0.6 g/100 mL to about 1.25 g/100 mL, from about 0.7 g/100 mL to about 1.25 g/100 mL, from about 0.8 g/100 mL to about 1.25 g/100 mL, from about 0.9 g/100 mL to about 1.25 g/100 mL, from about 1.0 g/100 mL to about 1.25 g/100 mL, from about 1.1 g/100 mL to about 1.25 g/100 mL, or from about 1.2 g/100 mL to about 1.25 g/100 mL. In embodiments, the lecithin is a sunflower lecithin and present at concentrations of any of the aforementioned values or ranges.

The aqueous phase may also include a preservative. Suitable preservatives used in some of the embodiments of present disclosure include, but are not limited to, disodium EDTA, tocopherol, butylated hydroxyanisole (BHA), ascorbic acid, butylated hydroxytoluene (BHT), or combinations thereof. In embodiments, the preservative can be present in the emulsion at a concentration of from about 0.001 to about 0.1 g/100 mL, including about 0.001 g/100 mL, about 0.002 g/100 mL, about 0.003 g/100 mL, about 0.004 g/100 mL, about 0.005 g/100 mL, about 0.006 g/100 mL, about 0.007 g/100 mL, about 0.008 g/100 mL, about 0.009 g/100 mL, about 0.01 g/100 mL, about 0.011 g/100 mL, about 0.012 g/100 mL, about 0.013 g/100 mL, about 0.014 g/100 mL, about 0.015 g/100 mL, about 0.017 g/100 mL, about 0.018 g/100 mL, about 0.019 g/100 mL, about 0.02 g/100 mL, about 0.025 g/100 mL, about 0.03 g/100 mL, about 0.035 g/100 mL, about 0.04 g/100 mL, about 0.045 g/100 mL, about 0.05 g/100 mL, about 0.055 g/100 mL, about 0.06 g/100 mL, about 0.065 g/100 mL, about 0.07 g/100 mL, about 0.075 g/100 mL, about 0.08 g/100 mL, about 0.085 g/100 mL, about 0.09 g/100 mL, about 0.095 g/100 mL, about 0.1 g/100 mL, or any range having endpoints defined by any two of the aforementioned values.

In embodiments, the concentration of preservative in the emulsion ranges from about 0.001 g/100 mL to about 0.1 g/100 mL, from about 0.002 g/100 mL to about 0.1 g/100 mL, from about 0.003 g/100 mL to about 0.1 g/100 mL, from about 0.004 g/100 mL to about 0.1 g/100 mL, from about 0.005 g/100 mL to about 0.1 g/100 mL, from about 0.006 g/100 mL to about 0.1 g/100 mL, from about 0.007 g/100 mL to about 0.1 g/100 mL, from about 0.008 g/100 mL to about 0.1 g/100 mL, from about 0.009 g/100 mL to about 0.1 g/100 mL, from about 0.01 g/100 mL to about 0.1 g/100 mL, from about 0.02 g/100 mL to about 0.1 g/100 mL, from about 0.03 g/100 mL to about 0.1 g/100 mL, from about 0.04 g/100 mL to about 0.1 g/100 mL, from about 0.05 g/100 mL to about 0.1 g/100 mL, from about 0.06 g/100 mL to about 0.1 g/100 mL, from about 0.07 g/100 mL to about 0.1 g/100 mL, from about 0.08 g/100 mL to about 0.1 g/100 mL, from about 0.09 g/100 mL to about 0.1 g/100 mL, from about 0.001 g/100 mL to about 0.01 g/100 mL, from about 0.002 g/100 mL to about 0.01 g/100 mL, from about 0.003 g/100 mL to about 0.01 g/100 mL, from about 0.004 g/100 mL to about 0.01 g/100 mL, from about 0.005 g/100 mL to about 0.01 g/100 mL, from about 0.006 g/100 mL to about 0.01 g/100 mL, from about 0.007 g/100 mL to about 0.01 g/100 mL, from about 0.008 g/100 mL to about 0.01 g/100 mL, from about 0.009 g/100 mL to about 0.01 g/100 mL, from about 0.01 g/100 mL to about 0.02 g/100 mL, from about 0.011 g/100 mL to about 0.02 g/100 mL, from about 0.012 g/100 mL to about 0.02 g/100 mL, from about 0.013 g/100 mL to about 0.02 g/100 mL, from about 0.014 g/100 mL to about 0.015 g/100 mL, from about 0.016 g/100 mL to about 0.02 g/100 mL, from about 0.017 g/100 mL to about 0.02 g/100 mL, from about 0.018 g/100 mL to about 0.02 g/100 mL, from about 0.019 g/100 mL to about 0.02 g/100 mL, from about 0.01 g/100 mL to about 0.011 g/100 mL, from about 0.01 g/100 mL to about 0.012 g/100 mL, from about 0.01 g/100 mL to about 0.013 g/100 mL, from about 0.01 g/100 mL to about 0.014 g/100 mL, from about 0.01 g/100 mL to about 0.015 g/100 mL, from about 0.01 g/100 mL to about 0.016 g/100 mL, from about 0.01 g/100 mL to about 0.017 g/100 mL, from about 0.01 g/100 mL to about 0.018 g/100 mL, or from about 0.01 g/100 mL to about 0.019 g/100 mL. In embodiments, the preservative is disodium EDTA and is present in the emulsion at any of the aforementioned concentrations or ranges.

In embodiments, the aqueous phase also contains a pH buffer to promote stability of the emulsion formulation. Possible chemical degradation within lipid emulsions includes oxidation of unsaturated fatty acid residues, and hydrolysis of phospholipids leading to the formation of free fatty acids (FFA) and lysophospholipids. This degradation can affect pH, thereby leading to further degradation. Thus, pH should be controlled during manufacture and emulsion formulations may include a buffering agent to provide additional control. In embodiments, a pH buffer may be is an amino acid buffer, for example, alanine, aspartic acid, glycine, histidine, isoleucine, leucine, methionine, phenylalanine, proline, serine, valine, or mixtures thereof. The combination of a preservative and an amino acid buffer may serve as an antioxidant to protect unsaturated fatty acids.

Generally, the organic phase refers to the components in the formulation that individually exceed their solubility limit in the water phase. These materials generally have solubility of less than 1% in distilled water; however, water phase components such as salts may influence the solubility of certain oils. The organic phase refers to the non-aqueous portion, and in an oil-in-water emulsion, the organic phase is dispersed in droplets.

The organic phase includes an oil or a lipid. Lipids and oils may be derived from animals, plants, or petroleum and may be natural or synthetic (i.e., man-made, such as, but not limited to, silicones). In embodiments, the oil may be a vegetable oil. Vegetable oils often are used in clinical applications because of the presence of essential fatty acids, and in particular long-chain triglycerides (LCTs). LCTs are formed when three long chain fatty acids form ester bonds with the three hydroxyl groups on glycerol. Examples of vegetable oils include but are not limited to almond oil, canola oil, castor oil, coconut oil, corn oil, cottonseed oil, olive oil, peanut oil, safflower oil, soybean oil, sunflower oil, and sesame oil. In embodiments, hydrogenated or partially hydrogenated forms of these oils may also be included. In embodiments, the oil is soybean oil. Soybean oil contains a mixture of polyunsaturated fatty acids, mainly C₁₄, C₁₆, and C₁₈. In embodiments, the oil is olive oil. Olive oil contains mostly mono-unsaturated oleic acid. In embodiments, different oil bases, such as medium chain triglycerides (MCT) may also be included, especially with varying concentrations of the other ingredients and with different surfactants.

In embodiments, the concentration of oil in the emulsion ranges from about 1 g/100 mL to about 40 g/100 mL. In embodiments, the concentration of oil in the emulsion ranges from about 1 g/100 mL to about 40 g/100 mL, from about 5 g/100 mL to about 40 g/100 mL, from about 10 g/100 mL to about 40 g/100 mL, from about 15 g/100 mL to about 40 g/100 mL, from about 20 g/100 mL to about 40 g/100 mL, from about 25 g/100 mL to about 40 g/100 mL, from about 30 g/100 mL to about 40 g/100 mL, from about 35 g/100 mL to about 40 g/100 mL, from about 5 g/100 mL to about 35 g/100 mL, from about 10 g/100 mL to about 35 g/100 mL, from about 15 g/100 mL to about 35 g/100 mL, from about 20 g/100 mL to about 35 g/100 mL, from about 25 g/100 mL to about 35 g/100 mL, from about 30 g/100 mL to about 35 g/100 mL, from about 5 g/100 mL to about 30 g/100 mL, from about 10 g/100 mL to about 30 g/100 mL, from about 15 g/100 mL to about 30 g/100 mL, from about 20 g/100 mL to about 30 g/100 mL, from about 25 g/100 mL to about 30 g/100 mL, from about 5 g/100 mL to about 25 g/100 mL, from about 10 g/100 mL to about 25 g/100 mL, from about 15 g/100 mL to about 25 g/100 mL, from about 20 g/100 mL to about 25 g/100 mL, from about 5 g/100 mL to about 20 g/100 mL, from about 10 g/100 mL to about 20 g/100 mL, from about 15 g/100 mL to about 20 g/100 mL, from about 5 g/100 mL to about 15 g/100 mL, from about 10 g/100 mL to about 15 g/100 mL, or from about 5 g/100 mL to about 10 g/100 mL. In embodiments, the concentration of oil in the emulsions is about 1 g/100 mL, about 2 g/100 mL, about 3 g/100 mL, about 4 g/100 mL, about 5 g/100 mL, about 6 g/100 mL, about 7 g/100 mL, about 8 g/100 mL, about 9 g/100 mL, about 10 g/100 mL, about 11 g/100 mL, about 12 g/100 mL, about 13 g/100 mL, about 14 g/100 mL, about 15 g/100 mL, about 16 g/100 mL, about 17 g/100 mL, about 18 g/100 mL, about 19 g/100 mL, about 20 g/100 mL, about 21 g/100 mL, about 22 g/100 mL, about 23 g/100 mL, about 24 g/100 mL, about 25 g/100 mL, about 26 g/100 mL, about 27 g/100 mL, about 28 g/100 mL, about 29 g/100 mL, about 30 g/100 mL, about 31 g/100 mL, about 32 g/100 mL, about 33 g/100 mL, about 34 g/100 mL, about 35 g/100 mL, about 36 g/100 mL, about 37 g/100 mL, about 38 g/100 mL, about 39 g/100 mL, about 40 g/100 mL, or any range having endpoints defined by any two of the aforementioned values. In embodiments, the oil is selected from soybean or olive oil and is present at a concentration of any of the aforementioned values or ranges.

The organic phase may further comprise a co-surfactant. A co-surfactant may enhance electrostatic surface charge on the dispersed droplets, strengthen the interfacial film between oil and water, and/or improve droplet stability. Examples of suitable co-surfactants include, but are not limited to, sodium deoxycholate, sorbitan monolaurate, sorbitan monostearate, sorbitan tristearate, polysorbate 20, polysorbate 40, polysorbate 60, polysorbate 80, and poloxamers. In embodiments, the co-surfactant is present in the emulsion a concentration from about 0.1 to about 10 g/100 mL, including about 0.1 g/100 mL, about 0.2 g/100 mL, about 0.3 g/100 mL, about 0.4 g/100 mL, about 0.5 g/100 mL, about 0.6 g/100 mL, about 0.7 g/100 mL, about 0.8 g/100 mL, about 0.9 g/100 mL, about 1 g/100 mL, about 1.1 g/100 mL, about 1.2 g/100 mL, about 1.3 g/100 mL, about 1.4 g/100 mL, about 1.5 g/100 mL, about 1.6 g/100 mL, about 1.7 g/100 mL, about 1.8 g/100 mL, about 1.9 g/100 mL, about 2 g/100 mL, about 2.1 g/100 mL, about 2.2 g/100 mL, about 2.3 g/100 mL, about 2.4 g/100 mL, about 2.5 g/100 mL, about 2.6 g/100 mL, about 2.7 g/100 mL, about 2.8 g/100 mL, about 2.9 g/100 mL, about 3 g/100 mL, about 3.1 g/100 mL, about 3.2 g/100 mL, about 3.3 g/100 mL, about 3.4 g/100 mL, about 3.5 g/100 mL, about 3.6 g/100 mL, about 3.7 g/100 mL, about 3.8 g/100 mL, about 3.9 g/100 mL, about 4 g/100 mL, about 4.1 g/100 mL, about 4.2 g/100 mL, about 4.3 g/100 mL, about 4.4 g/100 mL, about 4.5 g/100 mL, about 4.6 g/100 mL, about 4.7 g/100 mL, about 4.8 g/100 mL, about 4.9 g/100 mL, about 5 g/100 mL, about 5.1 g/100 mL, about 5.2 g/100 mL, about 5.3 g/100 mL, about 5.4 g/100 mL, about 5.5 g/100 mL, about 5.6 g/100 mL, about 5.7 g/100 mL, about 5.8 g/100 mL, about 5.9 g/100 mL, about 6 g/100 mL, about 6.1 g/100 mL, about 6.2 g/100 mL, about 6.3 g/100 mL, about 6.4 g/100 mL, about 6.5 g/100 mL, about 6.6 g/100 mL, about 6.7 g/100 mL, about 6.8 g/100 mL, about 6.9 g/100 mL, about 7 g/100 mL, about 7.1 g/100 mL, about 7.2 g/100 mL, about 7.3 g/100 mL, about 7.4 g/100 mL, about 7.5 g/100 mL, about 7.6 g/100 mL, about 7.7 g/100 mL, about 7.8 g/100 mL, about 7.9 g/100 mL, about 8 g/100 mL, about 8.1 g/100 mL, about 8.2 g/100 mL, about 8.3 g/100 mL, about 8.4 g/100 mL, about 8.5 g/100 mL, about 8.6 g/100 mL, about 8.7 g/100 mL, about 8.8 g/100 mL, about 8.9 g/100 mL, about 9 g/100 mL, about 9.1 g/100 mL, about 9.2 g/100 mL, about 9.3 g/100 mL, about 9.4 g/100 mL, about 9.5 g/100 mL, about 9.6 g/100 mL, about 9.7 g/100 mL, about 9.8 g/100 mL, about 9.9 g/100 mL, about 10 g/100 mL, or any range having endpoints defined by any two of the aforementioned values.

In embodiments, the concentration of co-surfactant in the emulsion ranges from about 0.1 g/100 mL to about 10 g/100 mL, from about 0.4 g/100 mL to about 10 g/100 mL, from about 0.5 g/100 mL to about 10 g/100 mL, from about 1 g/100 mL to about 10 g/100 mL, from about 1.5 g/100 mL to about 10 g/100 mL, from about 2.0 g/100 mL to about 10 g/100 mL, from about 2.5 g/100 mL to about 10 g/100 mL, from about 3.0 g/100 mL to about 10 g/100 mL, from about 3.5 g/100 mL to about 10 g/100 mL, from about 4.0 g/100 mL to about 10 g/100 mL, from about 4.5 g/100 mL to about 10 g/100 mL, from about 5.0 g/100 mL to about 10 g/100 mL, from about 5.5 g/100 mL to about 10 g/100 mL, from about 6.0 g/100 mL to about 10 g/100 mL, from about 6.5 g/100 mL to about 10 g/100 mL, from about 7.0 g/100 mL to about 10 g/100 mL, from about 7.5 g/100 mL to about 10 g/100 mL, from about 8.0 g/100 mL to about 10 g/100 mL, from about 8.5 g/100 mL to about 10 g/100 mL, from about 9.0 g/100 mL to about 10 g/100 mL, from about 9.5 g/100 mL to about 10 g/100 mL, from about 0.1 g/100 mL to about 9.0 g/100 mL, from about 0.4 g/100 mL to about 9.0 g/100 mL, from about 0.5 g/100 mL to about 9.0 g/100 mL, from about 1 g/100 mL to about 9.0 g/100 mL, from about 1.5 g/100 mL to about 9.0 g/100 mL, from about 2.0 g/100 mL to about 9.0 g/100 mL, from about 2.5 g/100 mL to about 9.0 g/100 mL, from about 3.0 g/100 mL to about 9.0 g/100 mL, from about 3.5 g/100 mL to about 9.0 g/100 mL, from about 4.0 g/100 mL to about 9.0 g/100 mL, from about 4.5 g/100 mL to about 9.0 g/100 mL, from about 5.0 g/100 mL to about 9.0 g/100 mL, from about 5.5 g/100 mL to about 9.0 g/100 mL, from about 6.0 g/100 mL to about 9.0 g/100 mL, from about 6.5 g/100 mL to about 9.0 g/100 mL, from about 7.0 g/100 mL to about 9.0 g/100 mL, from about 7.5 g/100 mL to about 9.0 g/100 mL, from about 8.0 g/100 mL to about 9.0 g/100 mL, from about 8.5 g/100 mL to about 9.0 g/100 mL, from about 0.1 g/100 mL to about 8.8 g/100 mL, from about 0.4 g/100 mL to about 8.8 g/100 mL, from about 0.5 g/100 mL to about 8.8 g/100 mL, from about 1 g/100 mL to about 8.8 g/100 mL, from about 1.5 g/100 mL to about 8.8 g/100 mL, from about 2.0 g/100 mL to about 8.8 g/100 mL, from about 2.5 g/100 mL to about 8.8 g/100 mL, from about 3.0 g/100 mL to about 8.8 g/100 mL, from about 3.5 g/100 mL to about 8.8 g/100 mL, from about 4.0 g/100 mL to about 8.8 g/100 mL, from about 4.5 g/100 mL to about 8.8 g/100 mL, from about 5.0 g/100 mL to about 8.8 g/100 mL, from about 5.5 g/100 mL to about 8.8 g/100 mL, from about 6.0 g/100 mL to about 8.8 g/100 mL, from about 6.5 g/100 mL to about 8.8 g/100 mL, from about 7.0 g/100 mL to about 8.8 g/100 mL, from about 7.5 g/100 mL to about 8.8 g/100 mL, from about 8.0 g/100 mL to about 8.8 g/100 mL, or from about 8.5 g/100 mL to about 8.8 g/100 mL. In embodiments, the co-surfactant is polysorbate 80 and is present in the emulsion in any of the above-mentioned concentrations or ranges. In embodiments, the co-surfactant is sodium deoxycholate present in the emulsion at concentrations of from about 0.01 to about 10 g/100 mL, or any range having endpoints defined by any two of the aforementioned values.

Addition of a co-surfactant also may function to decrease the diameter of the particles. In embodiments, the lipid emulsion may have a mean particle size or droplet diameter of less than about 1000 nanometers. In particular, the lipid emulsion may have a mean particle or droplet diameter of less than about 500 nanometers. As used herein, the term “particle size” or “particle diameter” refers to the mean diameter of the particles in a sample, as measured by dynamic light scattering (DLS), multiangle light scattering (MALS), nanoparticle tracking analysis, or comparable techniques. It will be understood that a lipid emulsion as described herein will not be of uniform size but can be described by the average diameter and, optionally, the polydispersity index. In embodiments, the particle

In some embodiments, the lipid emulsions described herein can have an average particle diameter that is about 120 nm, 125 nm, 130 nm, 135 nm, 140 nm, 145 nm, 150 nm, 155 nm, 160 nm, 165 nm, 170 nm, 175 nm, 180 nm, 185 nm, 19 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285, nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385, nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485, nm, 490 nm, 495 nm, 500 nm, or any range having endpoints defined by any two of the aforementioned values. In some embodiments, lipid emulsions described herein have an average particle diameter between about 150 nm and about 500 nm, between about 150 nm and about 450 nm, between about 150 nm and about 400 nm, between about 150 nm and about 350 nm, between about 150 nm and about 300 nm, between about 150 nm and about 250 nm, between about 150 nm and about 200 nm as determined by DLS.

The organic phase, in embodiments, further comprises a cryogenic agent or cryoprotectant. A cryogenic agent provides freeze tolerance to an emulsion and improves the overall lubricating properties. Examples of cryogenic agents include glycerin, DMSO, ethylene glycol, propylene glycol, sucrose, and trehalose. Cryogenic agents may be present in the emulsion at a concentration of from about 0.1 to about 20 g/100 mL, including 0.1 to about 10 g/100 mL, including about 0.1 g/100 mL, about 0.2 g/100 mL, about 0.3 g/100 mL, about 0.4 g/100 mL, about 0.5 g/100 mL, about 0.6 g/100 mL, about 0.7 g/100 mL, about 0.8 g/100 mL, about 0.9 g/100 mL, about 1 g/100 mL, about 1.5 g/100 mL, about 2 g/100 mL, about 2.5 g/100 mL, about 3 g/100 mL, about 3.5 g/100 mL, about 4 g/100 mL, about 4.5 g/100 mL, about 5 g/100 mL, about 5.5 g/100 mL, about 6 g/100 mL, about 6.5 g/100 mL, about 7 g/100 mL, about 7.5 g/100 mL, about 8 g/100 mL, about 8.5 g/100 mL, about 9 g/100 mL, about 9.5 g/100 mL, about 10 g/100 mL, about 10.5 g/100 mL, about 11 g/100 mL, about 11.5 g/100 mL, about 12 g/100 mL, about 12.5 g/100 mL, about 13 g/100 mL, about 13.5 g/100 mL, about 14 g/100 mL, about 14.5 g/100 mL, about 15 g/100 mL, about 15.5 g/100 mL, about 16 g/100 mL, about 16.5 g/100 mL, about 17 g/100 mL, about 17.5 g/100 mL, about 18 g/100 mL, about 18.5 g/100 mL, about 19 g/100 mL, about 19.5 g/100 mL, or any range having endpoints defined by any two of the aforementioned values.

In embodiments, the concentration of cryogenic agent in the emulsion ranges from about 0.1 g/100 mL to about 20 g/100 mL, from about 0.2 g/100 mL to about 20 g/100 mL, from about 0.3 g/100 mL to about 20 g/100 mL, from about 0.4 g/100 mL to about 20 g/100 mL, from about 0.5 g/100 mL to about 20 g/100 mL, from about 1.0 g/100 mL to about 20 g/100 mL, from about 1.5 g/100 mL to about 20 g/100 mL, from about 2.0 g/100 mL to about 20 g/100 mL, from about 2.25 g/100 mL to about 20 g/100 mL, from about 2.5 g/100 mL to about 20 g/100 mL, from about 3.0 g/100 mL to about 20 g/100 mL, from about 3.5 g/100 mL to about 20 g/100 mL, from about 4.0 g/100 mL to about 20 g/100 mL, from about 4.5 g/100 mL to about 20 g/100 mL, from about 5.0 g/100 mL to about 20 g/100 mL, from about 6.0 g/100 mL to about 20 g/100 mL, from about 7.0 g/100 mL to about 20 g/100 mL, from about 8.0 g/100 mL to about 20 g/100 mL, from about 9.0 g/100 mL to about 20 g/100 mL, from about 10 g/100 mL to about 20 g/100 mL, from about 11 g/100 mL to about 20 g/100 mL, from about 12 g/100 mL to about 20 g/100 mL, from about 13 g/100 mL to about 20 g/100 mL, from about 14 g/100 mL to about 20 g/100 mL, from about 15 g/100 mL to about 20 g/100 mL, from about 16 g/100 mL to about 20 g/100 mL, from about 17 g/100 mL to about 20 g/100 mL, from about 18 g/100 mL to about 20 g/100 mL, from about 19 g/100 mL to about 20 g/100 mL, from about 0.1 g/100 mL to about 15 g/100 mL, from about 0.2 g/100 mL to about 15 g/100 mL, from about 0.3 g/100 mL to about 15 g/100 mL, from about 0.4 g/100 mL to about 15 g/100 mL, from about 0.5 g/100 mL to about 15 g/100 mL, from about 1.0 g/100 mL to about 15 g/100 mL, from about 1.5 g/100 mL to about 15 g/100 mL, from about 2.0 g/100 mL to about 15 g/100 mL, from about 2.25 g/100 mL to about 15 g/100 mL, from about 2.5 g/100 mL to about 15 g/100 mL, from about 3.0 g/100 mL to about 15 g/100 mL, from about 3.5 g/100 mL to about 15 g/100 mL, from about 4.0 g/100 mL to about 15 g/100 mL, from about 4.5 g/100 mL to about 15 g/100 mL, from about 5.0 g/100 mL to about 15 g/100 mL, from about 6.0 g/100 mL to about 15 g/100 mL, from about 7.0 g/100 mL to about 15 g/100 mL, from about 8.0 g/100 mL to about 15 g/100 mL, from about 9.0 g/100 mL to about 15 g/100 mL, from about 10 g/100 mL to about 15 g/100 mL, from about 11 g/100 mL to about 15 g/100 mL, from about 12 g/100 mL to about 15 g/100 mL, from about 13 g/100 mL to about 15 g/100 mL, from about 14 g/100 mL to about 15 g/100 mL, from about 0.1 g/100 mL to about 10 g/100 mL, from about 0.2 g/100 mL to about 10 g/100 mL, from about 0.3 g/100 mL to about 10 g/100 mL, from about 0.4 g/100 mL to about 10 g/100 mL, from about 0.5 g/100 mL to about 10 g/100 mL, from about 1.0 g/100 mL to about 10 g/100 mL, from about 1.5 g/100 mL to about 10 g/100 mL, from about 2.0 g/100 mL to about 10 g/100 mL, from about 2.25 g/100 mL to about 10 g/100 mL, from about 2.5 g/100 mL to about 10 g/100 mL, from about 3.0 g/100 mL to about 10 g/100 mL, from about 3.5 g/100 mL to about 10 g/100 mL, from about 4.0 g/100 mL to about 10 g/100 mL, from about 4.5 g/100 mL to about 10 g/100 mL, from about 5.0 g/100 mL to about 10 g/100 mL, from about 6.0 g/100 mL to about 10 g/100 mL, from about 7.0 g/100 mL to about 10 g/100 mL, from about 8.0 g/100 mL to about 10 g/100 mL, from about 9.0 g/100 mL to about 10 g/100 mL, from about 0.1 g/100 mL to about 5 g/100 mL, from about 0.2 g/100 mL to about 5 g/100 mL, from about 0.3 g/100 mL to about 5 g/100 mL, from about 0.4 g/100 mL to about 5 g/100 mL, from about 0.5 g/100 mL to about 5 g/100 mL, from about 1.0 g/100 mL to about 5 g/100 mL, from about 1.5 g/100 mL to about 5 g/100 mL, from about 2.0 g/100 mL to about 5 g/100 mL, from about 2.25 g/100 mL to about 5 g/100 mL, from about 2.5 g/100 mL to about 5 g/100 mL, from about 3.0 g/100 mL to about 5 g/100 mL, from about 3.5 g/100 mL to about 5 g/100 mL, from about 4.0 g/100 mL to about 5 g/100 mL, from about 4.5 g/100 mL to about 5 g/100 mL, from about 0.1 g/100 mL to about 2.25 g/100 mL, from about 0.2 g/100 mL to about 2.25 g/100 mL, from about 0.3 g/100 mL to about 2.25 g/100 mL, from about 0.4 g/100 mL to about 2.25 g/100 mL, from about 0.2.25 g/100 mL to about 2.25 g/100 mL, from about 1.0 g/100 mL to about 2.25 g/100 mL, from about 1.2.25 g/100 mL to about 2.25 g/100 mL, or from about 2.0 g/100 mL to about 2.25 g/100 mL. In embodiments, the cryogenic agent is glycerin and is present in any of the aforementioned concentrations or ranges.

In embodiments, the lipid emulsion may include a pH adjuster to enhance stability. Examples of pH adjusters include, but are not limited to, sodium hydroxide, potassium hydroxide, magnesium hydroxide, Tris, sodium carbonate, and sodium linoleate. In embodiments, the pH of the emulsion may be adjusted to from about 6 to about 10, including 6, 7, 8, 9, 10, or any range having endpoints defined by any two of the aforementioned values.

The lipid emulsion may be prepared by any suitable process including, but not limited to, preparing an aqueous phase of water and an emulsifier; preparing an organic phase of a water-immiscible organic solvent, a cryogenic agent; and a co-surfactant; and mixing the aqueous phase and the organic phase to form an emulsion. In embodiments, the lipid emulsion can be prepared by combining sunflower lecithin with a vegetable oil, a co-surfactant, a cryogenic agent, and water, and processed using homogenization techniques. Homogenization of the lipid emulsion can be accomplished using any appropriate technique or device, such as the NanoDeBEE Laboratory Homogenizer, however any suitable technique or device is contemplated and possible. In some embodiments, the flow rate, flow direction, operating pressure, nozzle size, and possessing time are all adjustable to customize cavitation, shear, and impact of the emulsion.

In embodiments, the homogenization device is set up using parallel flow, wherein the product flows through the nozzle in a single direction and exits the opposite end. In embodiments, the homogenization device is set up using reverse flow, where the product is forced to flow back creating an annular stream, generating high shear between the opposing streams. In embodiments, a dual flow set up creates two impinging jets of product by using to inlets and two nozzles.

Nozzles can be made from any suitable material, such as zirconia, and have any suitable size, such as 0.005 inches (0.13 mm) diameter or 0.008 inches (0.2 mm) diameter, though any suitable nozzle material and size are contemplated and possible. Pressure used for homogenization can range from about 2,000 pounds per square inch (PSI) (13.8 MPa) to about 45,000 PSI, including about 3,000 PSI, about 4,000 PSI, about 5,000 PSI, about 6,000 PSI, about 7,000 PSI, about 8,000 PSI, about 9,000 PSI, about 10,000 PSI, about 15,000 PSI, about 15,000 PSI, about 25,000 PSI, about 30,000 PSI, about 35,000 PSI, about 40,000 PSI, about 45,000 PSI, or any range having endpoints defined by any two of the aforementioned values. In embodiments, pressure used for homogenization is from about 10,000 PSI to about 35,000 PSI.

In embodiments, the processing time can range from about 1 minute to about 10 hours, or from about 1 minute to about 60 minutes, including 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 15 minutes, 25 minutes, 30 minutes, 35 minutes, 40 minutes, 45 minutes, 50 minutes, 55 minutes, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, 5.75 hours, 6 hours, 6.25 hours, 6.5 hours, 6.75 hours, 7 hours, 7.25 hours, 7.5 hours, 7.75 hours, 8 hours, 8.25 hours, 8.5 hours, 8.75 hours, 9 hours, 9.25 hours, 9.5 hours, 9.75 hours, 10 hours, or any range having endpoints defined by any two of the aforementioned times.

In embodiments, the lipid emulsion is further diluted in a pharmaceutically acceptable fluid. Examples of pharmaceutically acceptable fluids include, but are not limited to, saline, Lactated Ringers, Ringers acetate, DSW, and DSNS. In embodiments, the emulsion is diluted to about 10% (v/v), about 9% (v/v), about 8% (v/v), about 7% (v/v), about 6% (v/v), about 5% (v/v), about 4% (v/v), about 3% (v/v), about 2% (v/v), about 1% (v/v), about 0.9% (v/v), about 0.8% (v/v), about 0.7% (v/v), about 0.6% (v/v), about 0.5% (v/v), about 0.4% (v/v), about 0.3% (v/v), about 0.2% (v/v), about 0.1% (v/v), about 0.09% (v/v), about 0.08% (v/v), about 0.07% (v/v), about 0.06% (v/v), about 0.05% (v/v), about 0.04% (v/v), about 0.03% (v/v), about 0.02% (v/v), about 0.01% (v/v), about 0.009% (v/v), about 0.008% (v/v), about 0.007% (v/v), about 0.006% (v/v), about 0.005% (v/v), about 0.004% (v/v), about 0.003% (v/v), about 0.002% (v/v), about 0.001% (v/v), or any range having endpoints defined by any two of the aforementioned values, for example, from about 10% (v/v) to about 0.001% (v/v), from about 10% (v/v) to about 0.01% (v/v), from about 10% (v/v) to about 0.1% (v/v), from about 10% (v/v) to about 1% (v/v), from about 10% (v/v) to about 2% (v/v), from about 10% (v/v) to about 3% (v/v), from about 10% (v/v) to about 4% (v/v), or from about 10% (v/v) to about 5% (v/v).

In embodiments, the emulsion is used as a lubricant for medical devices. Examples of suitable uses include, but are not limited to, use as a lubricant on endoscopic devices to ease movement in a lumen, use as a coating on sutures to ease friction, use as a lubricant for ultrasonic devices, use as a lubricant for devices that rotate, and use as a lubricant for devices that expand (e.g., balloon catheters). In embodiments, the emulsion is infused through the device and into a body lumen. The emulsion may be used also to lubricate the contact zone between a medical device and tissue, e.g., a vessel wall. In embodiments, the emulsion facilitates placement of a device in a lumen. For example, the emulsions disclosed herein are suitable for use during atherectomy procedures, angioplasty, and thrombectomy. In a particular embodiment, the emulsion is diluted with saline and intravenously infused through a catheter housing a rotary device, e.g., an atherectomy or ultrasonic drive shaft. In such embodiments, the emulsion lubricates the moving parts and enters the blood stream of a patient. Illustrative examples of medical devices that can be used with the lipid emulsions disclosed herein, include, but are not limited to, those devices disclosed in U.S. Pat. No. 10,646,248; International Patent Application Publication No. WO1521593281; International Patent Application Serial No. PCT/US1522/034210; U.S. Patent Application Publication No. US1590261516A1; U.S. Patent Application Publication No. US1550345380A1; U.S. Patent Application Publication No. US1590290303A1; and U.S. Patent Application Publication No. US15150261111A1.

In embodiments, the emulsions are used as nutritional formulations. In embodiments, the emulsions may provide enteral and/or parenteral nutrition to a subject. Illustrative, non-limiting examples of enteral administration include nasogastric tubes, nasojejunal tubes, percutaneous endoscopic gastrostomy tubes, gastrojejunal tubes, jejunal tubes, combinations thereof, and the like. Parenteral administration typically relies on intravenous therapy, though other administration routes are contemplated and possible. Illustrative, non-limiting examples of intravenous therapy include peripheral inserted catheters (PICC), central venous catheter, implanted ports, and the like. The lipid emulsions can be used with other sources of nutrition including protein solutions, carbohydrates, electrolytes, trace elements, vitamins, and/or other micronutrients.

In embodiments, the emulsion may be used as a dietary supplement. In embodiments, the emulsion may be formulated for oral administration. Illustrative examples of formulations for oral administration include, but are not limited to, tablets, capsules, lozenges, granules, and the like.

In embodiments, the emulsions may be employed as a drug delivery vehicle. In embodiments, the emulsion further comprises therapeutic agents, e.g., pharmaceuticals, and the like. The emulsion can be administered at a particular location to allow for targeted delivery of a therapeutic agent. Examples of therapeutics include, but are not limited to: anti-thrombogenic agents such as heparin, heparin derivatives, and urokinase; anti-proliferative agents such as enoxaprin, angiopeptin, paclitaxel, sirolimus or limus derivatives, sunitinib, nintedanib, sildenafil, tadalafil and acetylsalicylic acid; anti-inflammatory agents such as dexamethasone, prednisolone, corticosterone, budesonide, estrogen, sulfasalazine, colchicine, and mesalamine; antineoplastic/antiproliferative/anti-miotic agents such as endostatin, angiostatin and thymidine kinase inhibitors; anesthetic agents such as lidocaine, bupivacaine, and ropivacaine; anti-coagulants such as heparin, antithrombin compounds, platelet receptor antagonists, anti-thrombin antibodies, aspirin, prostaglandin inhibitors, platelet inhibitors; therapeutic polynucleotides; therapeutic polypeptides such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin like growth factor, and CD inhibitors; vascular cell growth promoters such as growth factor inhibitors, growth factor receptor antagonists, transcriptional activators of genes encoding vascular cell growth promoter proteins, and translational activators of mRNAs encoding vascular cell growth promoter proteins; vascular cell growth inhibitors such as growth factor inhibitors, growth factor receptor antagonists, transcriptional repressors of genes encoding vascular cell growth inhibitors, translational repressors of mRNAs encoding vascular cell growth inhibitors, DNA replication inhibitors, vascular cell growth inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; and tissue stabilization agents such as rose bengal, genipin, transglutaminase, and pentagallolyl glucose.

### EXAMPLES

The following examples are offered by way of illustration only. In view of the foregoing description, the person having ordinary skill in the art should recognize that the following examples are not intended to limit the scope of this disclosure or its many embodiments.

### Experimental Procedures

The chemicals are used without further purification unless noted otherwise. Particle size measurement and zeta potential data were recorded using a Brookhaven NanoBrook ZetaPALS analyzer.

ViperSlide® lubricant, commercially available from Cardiovascular Systems, Inc., is used for comparison. ViperSlide contains 10% (wt/vol) soybean oil, 1.2% (wt/vol) egg yolk lecithin, 2.25% (wt/vol) glycerin and is pH adjusted with sodium hydroxide. ViperSlide generated a mean particle size of 241 nm and a zeta potential between −58.97 mV and −60.15 mV.

### Characterization:

### Particle Size Measurement/Distribution

Dynamic Light Scattering (DLS) technique measures motion optically by recording the scattered light signal at a fixed angle. Particle size can be calculated using the Stokes-Einstein relationship (equation 1).

$$D = kT / 3 πη {d}_{p} \qquad ( 1 )$$

where k=Boltzmann constant, T=temperature, η=viscosity, dp'₂ hydrodynamic diameter, D=diffusion coefficient. Dynamic light scattering (DLS) studies are completed for various formulations, various concentrations, and various processing times. The concentration range for particle measurement for the tested formulations is from about 0.2% (v/v) to about 0.08% (v/v) Formulations with smaller mean particle diameter indicate a more stable emulsion. Particle diameter values for the tested formulations are less than 1000 nm, and in particular are less than 500 nm.

For each of the formulations tested, a Brookhaven NanoBrook ZetaPALS analyzer was used to run a particle analysis. The temperature was set to 25° C. The set duration time was 90 seconds. The equilibration time was 90 seconds. Five measurements were taken of each sample, with zero seconds between measurements. Unless otherwise indicated, particle size analysis was characterized using the aforementioned parameters.

### Zeta Potential

Zeta potential measures the electrochemical equilibrium at the particle-liquid interface (i.e. the slipping plane) and measures the magnitude of electrostatic interaction between particles. Emulsions with high zeta potential (±61 mV) are considered to have excellent stability. Zeta potentials are calculated using the electrophoreticmobility measurements from the ZetaPALS analyzer. Zeta potential can also be measured using electrophoresis, tunable resistive pulse sensing, or any other appropriate analytical technique.

### Visual Inspection

Formulations are visually inspected to monitor homogeneity, surface oil, creaming, color, opacity and other visual changes. Visual inspection takes place on initial preparation and at various time intervals after to measure stability.

### Additional Testing

Embodiments of the present disclosure may undergo additional testing. Additional types of testing may include functionality testing, where an emulsion is diluted in a pharmaceutically acceptable fluid and efficacy of the emulsion as a lubricant for medical devices is evaluated. This evaluation may include, but is not limited to, observations of kinking, twisting, jamming, and performance of the device.

Additional characterization of the emulsions is performed using any conventional and appropriate analytical technique. These characterizations include data collected at initial formulation and during stability analysis. The data collected may include, but is not limited to pH, osmolarity, osmolality, specific gravity, sterility testing, and stress testing. Testing recommended by any pharmacopeia, (i.e. the United States Pharmacopeia (USP), the European Pharmacopeia, the British Pharmacopeia, the Japanese Pharmacopeia, etc.) and any testing required by any regulatory agency, including the FDA, may also be completed.

### Example 1

A lipid emulsion is prepared according to the composition provided in Table 1.

TABLE 1
Lipid Emulsion Composition

| Ingredient | Function | W/V % |
|---|---|---|
| Soybean Oil | Oil | 10 |
| Sunflower Lecithin | Emulsifier | 1.2 |
| (Sunlipon ® 65) | | |
| Polysorbate 80 | Co-surfactant | 1 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 10 g soybean oil, 2.25 g glycerin, and 1 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for about 60 minutes. The pH is adjusted using NaOH. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, large oil droplets were present on the surface. After approximately one month of storage, a creamy layer formed and black gel-like particles were present in the container.

### Example 2

A lipid emulsion is prepared according to the composition provided in Table 2.

TABLE 2
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Sodium Deoxycholate | Co-surfactant | 0.4 |
| Sodium EDTA | Preservative | 0.01 |
| Glycerin | Cryogenic Agent | 5 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved. After dissolution, 0.01 g of sodium EDTA was added to the aqueous phase.

An organic phase is prepared by adding 15 g soybean oil, 5 g glycerin, and 0.4 g sodium deoxycholate to a 100 mL beaker and mixing with a spatula.

The organic phase and the aqueous phase are mixed by first placing the aqueous phase in a benchtop homogenizer and homogenizing for about 30-60 seconds at 5000 RPM. The organic phase is added dropwise via pipette into the aqueous phase while homogenizing at 10,000 RPM.

The mixture is homogenized at 10,000 RPM for 60 minutes. The pH is adjusted using NaOH. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion was separated into a creamy layer on top and slightly yellow aqueous phase on the bottom.

### Example 3

A lipid emulsion is prepared according to the composition provided in Table 3.

TABLE 3
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Sodium Deoxycholate | Co-surfactant | 0.4 |
| Sodium EDTA | Preservative | 0.01 |
| Glycerin | Cryogenic Agent | 10 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved. After dissolution, 0.01 g of sodium EDTA is added to the aqueous phase.

An organic phase is prepared by adding 15 g soybean oil, 10 g glycerin, and 0.4 g sodium deoxycholate to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. The pH is adjusted using NaOH. 1-2 mL aliquots are removed from the emulsion at 10 minute intervals for particle size characterization. After homogenizing, the emulsion is transferred to an appropriate container for storage.

### Example 4

A lipid emulsion is prepared according to the composition provided in Table 4.

TABLE 4
Lipid Emulsion Composition

| Ingredient | Function | W/V % |
|---|---|---|
| Olive Oil | Oil | 15 |
| Sunflower Lecithin | Emulsifier | 1.2 |
| (Sunlipon ® 65) | | |
| Sodium Deoxycholate | Co-surfactant | 0.4 |
| Sodium EDTA | Preservative | 0.01 |
| Glycerin | Cryogenic Agent | 5 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved. After dissolution, 0.01 g of sodium EDTA is added to the aqueous phase.

An organic phase is prepared by adding 15 g olive oil, 5 g glycerin, and 0.4 g sodium deoxycholate to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

### Example 5

A lipid emulsion is prepared according to the composition provided in Table 5.

TABLE 5
Lipid Emulsion Composition

| Ingredient | Function | W/V % |
|---|---|---|
| Olive Oil | Oil | 15 |
| Sunflower Lecithin | Emulsifier | 1.2 |
| (Sunlipon ® 65) | | |
| Sodium Deoxycholate | Co-surfactant | 0.4 |
| Sodium EDTA | Preservative | 0.01 |
| Glycerin | Cryogenic Agent | 10 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved. After dissolution, 0.01 g of sodium EDTA is added to the aqueous phase.

An organic phase is prepared by adding 15 g olive oil, 10 g glycerin, and 0.4 g sodium deoxycholate to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

### Example 6

A lipid emulsion is prepared according to the composition provided in Table 6.

TABLE 6
Lipid Emulsion Composition

| Ingredient | Function | W/V % |
|---|---|---|
| Olive Oil | Oil | 15 |
| Sunflower Lecithin | Emulsifier | 1.2 |
| (Sunlipon ® 65) | | |
| Polysorbate 80 | Co-surfactant | 1 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 15 g olive oil, 2.25 g glycerin, and 1 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

### Example 7

A lipid emulsion is prepared according to the composition provided in Table 7.

TABLE 7
Lipid Emulsion Composition

| Ingredient | Function | W/V % |
|---|---|---|
| Olive Oil | Oil | 10 |
| Sunflower Lecithin | Emulsifier | 1.2 |
| (Sunlipon ® 65) | | |
| Polysorbate 80 | Co-surfactant | 1 |
| Glycerin | Cryogenic Agent | 5 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 10 g olive oil, 5 g glycerin, and 1 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

### Example 8

A lipid emulsion is prepared according to the composition provided in Table 8.

TABLE 8
Lipid Emulsion Composition

| Ingredient | Function | W/V % |
|---|---|---|
| Olive Oil | Oil | 10 |
| Sunflower Lecithin | Emulsifier | 1.2 |
| (Sunlipon ® 65) | | |
| Sodium Deoxycholate | Co-surfactant | 0.4 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 10 g olive oil, 2.25 g glycerin, and 0.4 g sodium deoxycholate to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

### Example 9

A lipid emulsion is prepared according to the composition provided in Table 9.

TABLE 9
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Glycerin | Cryogenic Agent | 5 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 15 g soybean oil and 5 g glycerin to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation and small oil droplets were observed. After one week, a clear oil layer was present.

### Example 10

A lipid emulsion is prepared according to the composition provided in Table 10.

TABLE 10
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Glycerin | Cryogenic Agent | 10 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 15 g soybean oil and 10 g glycerin to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation and small oil droplets were observed. After one week, the droplets appeared larger.

### Example 11

A lipid emulsion is prepared according to the composition provided in Table 11.

TABLE 11
Lipid Emulsion Composition

| Ingredient | Function | W/V % |
|---|---|---|
| Olive Oil | Oil | 10 |
| Sunflower Lecithin | Emulsifier | 1.2 |
| (Sunlipon ® 65) | | |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 10 g olive oil and 2.25 g glycerin to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

### Example 12

A lipid emulsion is prepared according to the composition provided in Table 12.

TABLE 12
Lipid Emulsion Composition

| Ingredient | Function | W/V % |
|---|---|---|
| Olive Oil | Oil | 10 |
| Sunflower Lecithin | Emulsifier | 1.2 |
| (Sunlipon ® 65) | | |
| Glycerin | Cryogenic Agent | 5 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 10 g olive oil and 5 g glycerin to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

### Example 13

A lipid emulsion is prepared according to the composition provided in Table 13.

TABLE 13
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Polysorbate 80 | Co-surfactant | 1 |
| Glycerin | Cryogenic Agent | 5 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 15 g soybean oil, 5 g glycerin, and 1 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation, but did have a creamy layer on top. Small oil droplets were observed. After one week, the emulsion remained unchanged.

### Example 14

A lipid emulsion is prepared according to the composition provided in Table 14.

TABLE 14
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Polysorbate 80 | Co-surfactant | 1 |
| Glycerin | Cryogenic Agent | 10 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 15 g soybean oil, 10 g glycerin, and 1 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation, but did have a creamy layer on top. Small oil droplets were observed. After one week, the emulsion remained unchanged.

### Example 15

A lipid emulsion is prepared according to the composition provided in Table 15.

TABLE 15
Lipid Emulsion Composition

| Ingredient | Function | W/V % |
|---|---|---|
| Soybean Oil | Oil | 10 |
| Sunflower Lecithin | Emulsifier | 1.2 |
| (Sunlipon ® 65) | | |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 10 g soybean oil and 2.25 g glycerin to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation, but did have a slight creamy layer on top. Oil droplets were observed. After five days, phase separation began to occur and the oil droplets became larger.

### Example 16

A lipid emulsion is prepared according to the composition provided in Table 16.

TABLE 16
Lipid Emulsion Composition

| Ingredient | Function | W/V % |
|---|---|---|
| Soybean Oil | Oil | 10 |
| Sunflower Lecithin | Emulsifier | 1.2 |
| (Sunlipon ® 65) | | |
| Polysorbate 80 | Co-surfactant | 3.8 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 10 g soybean oil, 2.25 g glycerin, and 3.8 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation, but did have a slight creamy layer on top. No oil droplets were observed.

This exemplary formulation generated a mean particle size of 468 nm and a zeta potential between −74.16 mV and −81.07 mV.

### Example 17

A lipid emulsion is prepared according to the composition provided in Table 17.

TABLE 17
Lipid Emulsion Composition

| Ingredient | Function | W/V % |
|---|---|---|
| Soybean Oil | Oil | 10 |
| Sunflower Lecithin | Emulsifier | 1.2 |
| (Sunlipon ® 65) | | |
| Polysorbate 80 | Co-surfactant | 6.3 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 60° C. for about 40-45 minutes until the sunflower lecithin is fully dissolved.

An organic phase is prepared by adding 10 g soybean oil, 2.25 g glycerin, and 6.3 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation, but did have a slight creamy layer on top. No oil droplets were observed.

This exemplary formulation generated a mean particle size of 271 nm.

### Example 18

A lipid emulsion is prepared according to the composition provided in Table 18.

TABLE 18
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Polysorbate 80 | Co-surfactant | 8.8 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 60° C. for about 40-45 minutes until the sunflower lecithin is fully dissolved.

An organic phase is prepared by adding 15 g soybean oil, 2.25 g glycerin, and 8.8 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation, but did have a slight creamy layer on top. No oil droplets were observed.

### Example 19

A lipid emulsion is prepared according to the composition provided in Table 19.

TABLE 19
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Polysorbate 80 | Co-surfactant | 8.8 |
| Glycerin | Cryogenic Agent | 5 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 15 g soybean oil, 5 g glycerin, and 8.8 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation, but did have a slight creamy layer on top. No oil droplets were observed.

This exemplary formulation generated a mean particle size of 108 nm and a zeta potential between −71.85 mV and −77.66 mV.

### Example 15

A lipid emulsion is prepared according to the composition provided in Table 15.

TABLE 15
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Sodium Deoxycholate | Co-surfactant | 0.4 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 15 g soybean oil, 2.25 g glycerin, and 0.4 g sodium deoxycholate to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

### Example 21

A lipid emulsion is prepared according to the composition provided in Table 21.

TABLE 21
Lipid Emulsion Composition

| Ingredient | Function | W/V % |
|---|---|---|
| Soybean Oil | Oil | 15 |
| Sunflower Lecithin | Emulsifier | 1.2 |
| (Sunlipon ® 65) | | |
| Polysorbate 80 | Co-surfactant | 6.3 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 15 g soybean oil, 2.25 g glycerin, and 6.3 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. On initial inspection, the emulsion had no phase separation, but did have a slight creamy layer on top. No oil droplets were observed.

### Example 22

A lipid emulsion is prepared according to the composition provided in Table 22.

TABLE 22
Lipid Emulsion Composition

| Ingredient | Function | W/V % |
|---|---|---|
| Soybean Oil | Oil | 10 |
| Sunflower Lecithin | Emulsifier | 1.2 |
| (Sunlipon ® 50) | | |
| Polysorbate 80 | Co-surfactant | 6.3 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 50° C. for about 40-45 minutes until the sunflower lecithin is fully dissolved.

An organic phase is prepared by adding 10 g soybean oil, 2.25 g glycerin, and 6.3 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. This exemplary formulation generated a mean particle size of 163 nm.

### Example 23

A lipid emulsion is prepared according to the composition provided in Table 23.

TABLE 23
Lipid Emulsion Composition

| Ingredient | Function | W/V % |
|---|---|---|
| Soybean Oil | Oil | 10 |
| Sunflower Lecithin | Emulsifier | 1.2 |
| (Sunlipon ® 90) | | |
| Polysorbate 80 | Co-surfactant | 6.3 |
| Glycerin | Cryogenic Agent | 2.25 |
| Water | Water | 100 mL |

An aqueous phase is prepared by stirring 1.2 g sunflower lecithin into 100 mL reverse osmosis water at 700 rpm at 60° C. for about 40-45 minutes until the sunflower lecithin is fully dissolved.

An organic phase is prepared by adding 10 g soybean oil, 2.25 g glycerin, and 6.3 g polysorbate 80 to a 100 mL beaker and mixing with a spatula.

The organic phase and the aqueous phase are mixed by first placing the aqueous phase in a benchtop homogenizer and homogenizing for about 30-60 seconds. The organic phase is added dropwise via pipette into the aqueous phase while homogenizing at 10,000 RPM.

The mixture is homogenized at 10,000 RPM for 60 minutes. 1-2 mL aliquots are removed from the emulsion at 10 minute intervals for particle size characterization. After homogenizing, the emulsion is transferred to an appropriate container for storage.

An emulsion was prepared according to the foregoing procedure. This exemplary formulation generated a mean particle size of 238 nm.

### Example 24

A lipid emulsion is prepared according to the composition provided in Table 24.

TABLE 24
Lipid Emulsion Composition

| Ingredient | Function | W/V % |
|---|---|---|
| Soybean Oil | Oil | 10 |
| Sunflower Lecithin | Emulsifier | 1.2 |
| (Sunlipon ® 65) | | |
| Polysorbate 80 | Co-surfactant | 6.3 |
| Glycerin | Cryogenic Agent | 2.3 |
| EDTA | Preservative | 0.017 |
| Water | Water | 1000 mL |

An aqueous phase is prepared by stirring 12 g sunflower lecithin into 1000 mL reverse osmosis water at 750 rpm at 50° C. for approximately 1 hour until the sunflower lecithin is fully dissolved.

An organic phase is prepared by mixing 100 g soybean oil, 23 g glycerin, and 63 g polysorbate 80 in a 150 mL beaker. The organic phase was added to the aqueous phase.

The emulsions were prepared using different processing conditions, including nozzles, operating pressures, and flow patterns, as described in Table 25.

TABLE 25
Lipid Emulsion Processing

| Sample | Nozzle | Pressure (PSI) × 1000 | Configuration |
|---|---|---|---|
| 1 | Z05 | 10 | Reverse |
| 2 | Z05 | 15 | Reverse |
| 3 | Z05 | 30 | Reverse |
| 4 | Z05 | 10 | Parallel |
| 5 | Z05 | 15 | Parallel |
| 6 | Z05 | 30 | Parallel |
| 7 | Z05 | 40 | Parallel |
| 8 | Z05 | 40 | Reverse |
| 9 | Z08 | 15 | Parallel |
| 10 | Z08 | 30 | Parallel |
| 11 | Z08 | 15 | Reverse |
| 12 | Z08 | 30 | Reverse |

Each of the samples were stored at 4.9° C. and tested biweekly for particle size. Additionally, samples 7 and 8 were stored at ambient temperature and tested biweekly for particle size. The results of these particle size tests are shown below for samples 1-8.

| | Diameter | | | | | | |
|---|---|---|---|---|---|---|---|
| Sample ID | 0 days | 14 days | 28 days | 42 days | 72 days | AVG | STDEV |
| 1 | 158.00 | 211.46 | 215.4 | 211.22 | 217.6 | 211.52 | 3.03 |
| 2 | 194.76 | 197.72 | 195.4 | 199.80 | 195.28 | 196.92 | 2.3 |
| 3 | 165.32 | 175.96 | 180.00 | 179.00 | 174.2 | 175.07 | 6.72 |
| 4 | 215.44 | 217.6 | 195.1 | 223.82 | 222.1 | 212.99 | 12.44 |
| 5 | 191.96 | 192.58 | 179.66 | 193.48 | 196.68 | 189.42 | 6.54 |
| 6 | 174.06 | 176.54 | 178.34 | 176.3 | 175.76 | 176.31 | 1.75 |
| 7 | 160.00 | 162.62 | 164.84 | 164.12 | 166.06 | 162.9 | 2.14 |
| 8 | 165.29 | 156.2 | 157.36 | 156.4 | 166.38 | 158.81 | 4.35 |
| 7 (ambient | 160.00 | 165.24 | 166.34 | 164.06 | 165.36 | 163.91 | 2.77 |
| temperature) | | | | | | | |
| 8 (ambient | 165.29 | 150.88 | 148.66 | 149.04 | 172.42 | 153.47 | 7.94 |
| temperature) | | | | | | | |

No statistically significant difference in particle diameter was observed for any time point or storage condition.

Visual inspection of the samples showed no phase separation at any time point or storage condition.

### Results:

Unexpectedly, the addition of co-surfactant, particularly polysorbate 80, demonstrated increased stability of the emulsion and prevention of coalescence. Polysorbate 80 stabilized droplets in emulsion for at least 7 days. In some examples, the addition of polysorbate 80 prevented coalescence and stabilized droplet size for at least 72 days. This impact was observed regardless of glycerin concentration, which was shown to have no impact on coalescence. The use of polysorbate 80 showed substantial stability over sodium deoxycholate. Increasing the concentration of polysorbate 80 reduced particle size, increased zeta potential, and minimized oil droplet coalescence.

### Items Listing

Embodiments of the present disclosure include at least following Items, which are not intended to limit the scope of the disclosure as a whole or the appended claims.

Item 1A: A lipid emulsion comprising: from about 1 g/100 mL to about 40 g/100 mL oil; from about 0.3 g/100 mL to about 3 g/100 mL sunflower lecithin; from about 0.1 g/100 mL to about 10 g/100 mL co-surfactant; from about 0.1 g/100 mL to about 10 g/100 mL cryogenic agent; and an aqueous liquid, wherein: the oil is selected from almond oil, canola oil, castor oil, corn oil, cottonseed oil, olive oil, peanut oil, safflower oil, soybean oil, sunflower oil, sesame oil, or mixtures thereof; the co-surfactant is selected from sodium deoxycholate, sorbitan monolaurate, sorbitan monostearate, sorbitan tristearate, polysorbate 15, polysorbate 40, polysorbate 60, polysorbate 80, poloxamers, or mixtures thereof; and the cryogenic agent is selected from glycerin, DMSO, ethylene glycol, propylene glycol, sucrose, trehalose, or mixtures thereof.

Item A2: The lipid emulsion of item A1, further comprising from about 0.001 g/100 mL to about 0.1 g/100 mL of an antimicrobial preservative selected from disodium EDTA, tocopherol, butylated hydroxyanisole (BHA), ascorbic acid, butylated hydroxytoluene (BHT), or a mixture thereof.

Item A3: The lipid emulsion of items A1 or A2, wherein the oil is olive oil, soybean oil, or a mixture thereof.

Item A4: The lipid emulsion of any of the preceding items, wherein the co-surfactant is sodium deoxycholate, polysorbate 80, or mixtures thereof, or wherein the co-surfactant is polysorbate 80.

Item A5: The lipid emulsion of any of the preceding items, wherein the cryogenic agent comprises glycerin.

Item A6: The lipid emulsion of any of the preceding items, comprising from about 6 g/100 mL to about 6.5 g/100 mL co-surfactant.

Item A7: The lipid emulsion of any of the preceding items, wherein the aqueous liquid comprises a pharmaceutically acceptable fluid.

Item A8: The lipid emulsion of item A1, wherein the lipid emulsion has a mean particle diameter of less than 1000 nm.

Item A9: A method of preparing a lipid emulsion, the method comprising: dissolving sunflower lecithin in water to create an aqueous phase; mixing an oil, a co-surfactant, and a cryogenic agent to create an organic phase; and homogenizing the aqueous phase and the organic phase to produce the lipid emulsion, wherein: the lipid emulsion comprises: from about 1 g/100 mL to about 40 g/100 mL of the oil; from about 0.3 g/100 mL to about 3 g/100 mL of the sunflower lecithin; from about 0.1 g/100 mL to about 10 g/100 mL of the co-surfactant; and from about 0.1 g/100 mL to about 10 g/100 mL of the cryogenic agent; the oil is selected from almond oil, canola oil, castor oil, corn oil, cottonseed oil, olive oil, peanut oil, safflower oil, soybean oil, sunflower oil, sesame oil, or mixtures thereof; the co-surfactant is selected from sodium deoxycholate, sorbitan monolaurate, sorbitan monostearate, sorbitan tristearate, polysorbate 15, polysorbate 40, polysorbate 60, polysorbate 80, poloxamers, or mixtures thereof; the cryogenic agent is selected from glycerin, DMSO, ethylene glycol, propylene glycol, sucrose, trehalose, or mixtures thereof; and the lipid emulsion has a mean particle diameter of less than 1000 nm.

Item A10: The method of item A9, wherein the oil is olive oil, soybean oil, or mixtures thereof.

Item A11: The method of item A9 or A10, wherein the co-surfactant is sodium deoxycholate, polysorbate 80, or mixtures thereof.

Item A12: The method of any of the previous items, wherein the cryogenic agent comprises glycerin.

Item A13: The method of any of the previous items, further comprising, adding an antimicrobial preservative to the lipid emulsion, wherein the antimicrobial preservative is selected from disodium EDTA, tocopherol, butylated hydroxyanisole (BHA), ascorbic acid, and butylated hydroxytoluene (BHT).

Item A14: A method administering a lipid emulsion to a subject, the method comprising: diluting a lipid emulsion according to any of the items A1 to A8 or a lipid emulsion made by any of the methods in items A9 to A13 in a pharmaceutically acceptable fluid; and parenterally administering the diluted emulsion to the subject.

Item A15: The method of item A14, wherein the pharmaceutically acceptable fluid is selected from saline, Lactated Ringers, Ringers acetate, D5W, and D5NS.

Item A16: The method of items A14 or A15, wherein the lipid emulsion is diluted to from about 10% (v/v) to about 0.001% (v/v).

Item A17: Use of a lipid emulsion according to any of items A1 to A8 or a lipid made by a method according to any of items A9 to A13 as a lubricant for a medical device.

Item A18: The use of item A18, wherein the medical device is an atherectomy device.

It should now be understood that embodiments of the present disclosure are directed to biocompatible and allergen-free lipid emulsions suitable for parenteral administration into a subject or patient, which include soybean lecithin, oil, a co-surfactant, and water. The co-surfactant may be polysorbate 80. The lipid emulsions are prepared by homogenizing organic and aqueous phases. In embodiments, the emulsions show increased stability, demonstrated by increased storage times before coalescence occurs, small particle size, and large zeta potential.

It is expressly contemplated that each of the various aspects, embodiments, and features thereof described herein may be freely combined with any or all other aspects, embodiments, and features. The resulting aspects and embodiments (e.g., formulations and methods) are within the scope of the invention. It should be understood that headings herein are provided for purposes of convenience and do not imply any limitation on content included below such heading or the use of such content in combination with content included below other headings.

Those skilled in the art will recognize, or be able to ascertain using no more than routine experimentation, many equivalents to the specific embodiments of the invention described and exemplified herein. The scope of the present invention is not intended to be limited to the above Detailed Description and Examples, but rather is as set forth in the appended claims.

1. A lipid emulsion comprising:
from about 1 g/100 mL to about 40 g/100 mL oil;
from about 0.3 g/100 mL to about 3 g/100 mL sunflower lecithin;
from about 0.1 g/100 mL to about 10 g/100 mL co-surfactant;
from about 0.1 g/100 mL to about 10 g/100 mL cryogenic agent; and
an aqueous liquid,
wherein:
the oil is selected from soybean oil, olive oil, almond oil, canola oil, castor oil, corn oil, cottonseed oil, peanut oil, safflower oil, sunflower oil, sesame oil, or mixtures thereof;
the co-surfactant is selected from polysorbate 80, sodium deoxycholate, sorbitan monolaurate, sorbitan monostearate, sorbitan tristearate, polysorbate 15, polysorbate 40, polysorbate 60, poloxamers, or mixtures thereof; and
the cryogenic agent is selected from glycerin, DMSO, ethylene glycol, propylene glycol, sucrose, trehalose, or mixtures thereof.

2. The lipid emulsion of claim 1, further comprising from about 0.001 g/100 mL to about 0.1 g/100 mL of an antimicrobial preservative selected from disodium EDTA, tocopherol, butylated hydroxyanisole (BHA), ascorbic acid, butylated hydroxytoluene (BHT), or a mixture thereof.

3. The lipid emulsion of claim 1, wherein the oil is olive oil, soybean oil, or a mixture thereof.

4. The lipid emulsion of claim 1, wherein the co-surfactant is sodium deoxycholate, polysorbate 80, or mixtures thereof.

5. The lipid emulsion of claim 1, wherein the co-surfactant is polysorbate 80.

6. The lipid emulsion of claim 1, wherein the cryogenic agent comprises glycerin.

7. The lipid emulsion of claim 1, comprising from about 6 g/100 mL to about 6.5 g/100 mL co-surfactant.

8. The lipid emulsion of claim 1, wherein the aqueous liquid comprises a pharmaceutically acceptable fluid.

9. The lipid emulsion of claim 1, wherein the lipid emulsion has a mean particle diameter of less than 1000 nm.

10. A method of preparing a lipid emulsion, the method comprising:
(i) dissolving sunflower lecithin in water to create an aqueous phase;
(ii) mixing an oil, a co-surfactant, and a cryogenic agent to create an organic phase; and
(iii) homogenizing the aqueous phase and the organic phase to produce the lipid emulsion,
wherein:
the lipid emulsion comprises:
from about 1 g/100 mL to about 40 g/100 mL of the oil;
from about 0.3 g/100 mL to about 3 g/100 mL of the sunflower lecithin;
from about 0.1 g/100 mL to about 10 g/100 mL of the co-surfactant; and
from about 0.1 g/100 mL to about 10 g/100 mL of the cryogenic agent;
the oil is selected from sunflower oil, olive oil, almond oil, canola oil, castor oil, corn oil, cottonseed oil, peanut oil, safflower oil, soybean oil, sesame oil, or mixtures thereof;
the co-surfactant is selected from polysorbate 80, sodium deoxycholate, sorbitan monolaurate, sorbitan monostearate, sorbitan tristearate, polysorbate 15, polysorbate 40, polysorbate 60, poloxamers, or mixtures thereof; and
the cryogenic agent is selected from glycerin, DMSO, ethylene glycol, propylene glycol, sucrose, trehalose, or mixtures thereof.

11. The method of claim 10, wherein the oil is olive oil, soybean oil, or mixtures thereof.

12. The method of claim 10, wherein the co-surfactant is polysorbate 80, sodium deoxycholate, or mixtures thereof.

13. The method of claim 10, wherein the cryogenic agent comprises glycerin.

14. The method of claim 10, further comprising, adding an antimicrobial preservative to the lipid emulsion, wherein the antimicrobial preservative is selected from disodium EDTA, tocopherol, butylated hydroxyanisole (BHA), ascorbic acid, and butylated hydroxytoluene (BHT).

15. A method of administering a lipid emulsion to a subject, the method comprising:
diluting a lipid emulsion according to claim 1 in a pharmaceutically acceptable fluid; and
parenterally administering the diluted emulsion to the subject.

16. The method of claim 15, wherein the pharmaceutically acceptable fluid is selected from saline, Lactated Ringers, Ringers acetate, D5W, and D5NS.

17. The method of claim 15, wherein the lipid emulsion is diluted in the pharmaceutically acceptable fluid to from about 10% (v/v) to about 0.001% (v/v).

18-19. (canceled)
